(19) United States
(12) Patent Application Publication
(10) Pub. No.: US 2024/0100158 A1
(43) Pub. Date: Mar. 28, 2024

(54) FREEZE DRIED ANTIBODY FORMULATIONS AND METHODS THEREOF

(71) Applicant: DR. REDDY’S LABORATORIES LIMITED, Hyderabad (IN)

(72) Inventors: Murali JAYARAMAN, Kancheepuram (Dist) (IN); K Saisharan GOUD, Kamareddy (Dist) (IN); Anandkumar Jitendra bhai KHRISTI, Vadodara (IN)

(21) Appl. No.: 18/273,142

(22) PCT Filed: Jan. 20, 2022
(86) PCT No.: PCT/IN2022/050044

§ 371 Date: Jul. 19, 2023

(30) Foreign Application Priority Data
Jan. 20, 2021 (IN) 202141002843

(51) Int. Cl. A61K 39/395; A61K 9/19; A61K 47/18; A61K 47/26; C07K 16/28

(52) U.S. Cl. CPC A61K 39/39591; A61K 9/19; A61K 47/183; A61K 47/26; C07K 16/2839

(57) ABSTRACT

The present invention discloses an optimal lyophilization method to prepare a room-temperature-stable freeze-dried formulation of an anti-α4β7 antibody wherein the lyophilized anti-α4β7 antibody formulation obtained from the said method exhibits stability at room temperature for at least three months and reconstituted anti-α4β7 antibody formulation exhibits stability at room temperature at least for 24 hours.

### FIELD OF THE INVENTION

The present invention relates to therapeutic proteins, particularly integrin antibody formulations, and process of preparing the freeze-dried therapeutic integrin antibody formulations. In particular, the invention relates to process of making room-temperature stable reconstituted freeze-dried integrin antibody formulations.

### BACKGROUND

Over the past two decades, recombinant DNA technology has led to the commercialization of many proteins, particularly antibody therapeutics and fusion protein molecules.

Anti-integrin antibodies, which bind to integrins, such as α4 integrin and α4β7 integrin, are one among the approved antibody therapeutics that provides good prognosis in the treatment of inflammatory bowel diseases (eg. natalizumab and vedolizumab). Vedolizumab, is an anti-α4β7 integrin antibody, and currently is being marketed under the following trade name Entyvio® and is approved to treat inflammatory bowel diseases such as Crohn's disease and Ulcerative colitis. Entyvio® was initially approved as a lyophilized/freeze-dried formulation in May 2014 by the US Food and Drug Administration (FDA) and the European Medical Agency (EMA) and later on, in May, 2020 Entyvio® was also approved as a liquid formulation by EMA.

The shelf-life of lyophilized and liquid Entyviox formulations is 3 years and 2 years, respectively, under storage conditions such as at 2-8° C. Further, in-use stability of lyophilized formulations is less when reconstituted with water for injection to prepare liquid formulation which is ready for administration.

The European public assessment report of Entyvio®, details on shelf life of the Entyvio's stability in section 6.3. Verbatim from the section is as follows: “In-use stability of the reconstituted solution in the vial has been demonstrated for 8 hours at 2° C.-8° C. In-use stability of the diluted solution in sodium chloride 9 mg/mL (0.9%) solution for injection in infusion bag has been demonstrated for 12 hours at 20° C.-25° C. or 24 hours at 2° C.-8° C. The combined in-use stability of vedolizumab in the vial and infusion bag with sodium chloride 9 mg/mL (0.9%) solution for injection is a total of 12 hours at 20° C.-25° C. or 24 hours at 2° C.-8° C. A 24 hour period may include up to 8 hours at 2° C.-8° C. for reconstituted solution in the vial and up to 12 hours at 20° C.-25° C. for diluted solution in the infusion bag but the infusion bag must be stored in the refrigerator (2° C.-8° C.) for the rest of the 24 hour period”.

In addition, FDA label of Entyvio®, details on reconstitution time of Entyvio's lyophilized formulation in point number 4 of section 2.4 (reconstitution an dilution instructions), verbatim from the section as follows “Allow the solution to sit for up to 20 minutes at room temperature to allow for reconstitution and for any foam to settle; the vial can be swirled and inspected for dissolution during this time. If not fully dissolved after 20 minutes, allow another 10 minutes for dissolution. Do not use the vial if the drug product is not dissolved within 30 minutes.”

The stability of the formulated therapeutic protein is essential to maintain its functionality and potency. In particular, room-temperature stability of liquid or lyophilized formulation is especially important in storage, transport and distribution of therapeutic proteins.

However, formulation of therapeutic proteins, whether liquid or lyophilized, encompass in itself various complexities. Multiple factors such as choice of buffer, pH, excipients and process involved in the formulation of protein significantly influence largely the stability, shelf life and potency of the protein.

“Lyophilization” or “freeze-drying” is a process whereby the substance to be lyophilized, is first frozen and then the solvent removed by primary and secondary drying, this technique is frequently employed as a formulation technique. Similar to selecting protein solvents and their components, excipients for lyophilization need to be carefully chosen so that the protein of interest is unaffected by the freezing process, and its stability not compromised during storage and subsequent use. It is also important to develop an optimized lyophilized cycle which can provide a stable, amorphous, less moisture containing cake. Further, a formulation (or lyophilized formulation) that can remain stable at room temperature, especially after reconstitution, would be highly advantageous.

Hence, the objective of the present invention to develop an optimized lyophilization process and stable formulation for therapeutic protein, in particular for therapeutic antibodies.

### SUMMARY

The present invention discloses a method for obtaining a freeze-dried formulation of vedolizumab, an anti-α4β7 antibody, by an optimized lyophilization process. The lyophilization process includes steps of, subjecting a liquid antibody composition to primary drying in a step-wise approach at different temperatures, wherein the antibody solution is subjected to a/first temperature which is glass transition temperature (Tg) of the antibody composition and holding at the glass transition temperature, followed by increasing temperatures to second and third temperatures and holding at the respective temperatures.

The freeze-dried product of vedolizumab obtained from the present invention is a solid, stable cake, which is free from collapse, and contains less than 1% of moisture content and requires lesser reconstitution time (solubilized or reconstituted in less than 10 minutes). Most importantly, the freeze-dried formulation of vedolizumab obtained from the said process remains stable at room-temperature (at 25° C.), for at least 24 hours, post reconstitution.

The disclosed lyophilization cycle does not impact the quality attributes of vedolizumab formulation pre and post-lyophilization process. Hence, aggregate content, main peak content, remains same.

The disclosed freeze-dried vedolizumab formulations of the invention are stable and contains less than 1% of the antibody in aggregate form when stored at 25° C. for three months, and less than 0.5% in aggregate form when stored at 2-8° C. for three months. The lower aggregate content (<0.5%) is maintained at least for 12 months when stored at 2-8° C.

Further, the reconstitution time of the freeze-dried powder, obtained from the said lyophilization process is less than 10 minutes and specifically, less than 5 minutes.

### DETAILED DESCRIPTION OF THE INVENTION

### Definitions

The terms “anti-α4β7 antibody” refers to an antibody which binds to α4β7 receptor. Vedolizumab is an anti-α4β7 antibody, and the sequence of this antibody is disclosed in U.S. Pat. No. 7,147,851.

The terms “cake” or “powder” are used synonymously herein, and refers to a dry pellet or powder that results when a liquid formulation has been lyophilized or freeze-dried. As used herein, “dry cake or powder” refers to a cake or powder that comprises about 1% or less residual moisture content. In some embodiments of the invention, the moisture content of the dry cake is about 0.1% to about 1%.

The term “stable” formulation refers to the formulation, wherein the Protein/antibody molecule therein retains its physical stability and/or chemical stability and/or biological activity, upon storage.

The term “Tg” refers to the glass transition temperature of the formulation or composition comprising various amorphous and/or crystalline components, and is the temperature at which the hard/glassy amorphous components in the formulation which has lower mobility starts converting to rubbery/soft state, in which the macromolecular mobility is higher. The term is majorly applicable for amorphous or amorphous part of semi crystalline components.

Ultrafiltration (UF) and diafiltration (DF) are commonly used steps in downstream processing for product concentration and buffer exchange. Ultrafiltration may be used to increase the concentration of macromolecules in a solution and diafiltration is generally used for buffer exchange. UF and DF steps mentioned herein can be either sequential or simultaneous. These filtration steps may be performed or operated in tangential flow filtration or cross flow filtration mode or normal (direct) flow filtration mode. Addition of maltose ‘at the time of lyophilization’ means addition performed after the UF/DF or Tangential flow filtration steps, but while subjecting to lyophilization process.

Stability studies provide evidence of the quality fusion protein under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the fusion protein in the pharmaceutical formulations. A fusion protein “retains its physical stability” in a pharmaceutical formulation if it shows substantially no signs of aggregation, precipitation and/or denaturation upon visual examination of color and/or clarity, or as measured by UV light scattering or by size exclusion chromatography. A fusion protein is said to “retain its chemical stability” in a pharmaceutical formulation when its shows no or minimal formation of product variants which may include variants as a result of chemical modification of fusion protein such as deamination, oxidation etc. Analytical methods such as ion exchange chromatography and hydrophobic ion chromatography may be used to investigate the chemical product variants.

The monomer, dimer and high molecular weight (HMW) species of α4β7 antibody molecule may be separated by size exclusion chromatography (SEC). SEC separates molecules based on the molecular size. Separation is achieved by the differential molecular exclusion or inclusion as the molecules migrate along the length of the column. Thus, resolution increases as a function of column length. In order to maintain the appropriate activity of a fusion protein, it is desirable to reduce the formation of aggregate or fragmentation (monomer/low molecular weight species) of products and hence control the dimer content to a target value. Dimer is major form present in fusion proteins and elutes as main peak in size exclusion chromatography. α4β7 antibody molecule samples may be separated using a 2695 Alliance HPLC (Waters, Milford, Mass.) equipped with TSK Gel® G3000SWXL (300 mm×7.8 mm) and TSK Gel® G3000SWXL (40 mm×6.0 mm) columns (Tosoh Bioscience, Montgomery, Pa.).

Pharmaceutically acceptable excipients refer to the additives or carriers, which may contribute to stability of the fusion protein in formulation. The excipients may encompass stabilizers and tonicity modifiers. Examples of stabilizers and tonicity modifiers include, but not limited to, sugars, salts, surfactants, and derivatives and combination thereof.

The term “reconstitution time” refers to the time taken to rehydrate or dissolve a dry lyophilized/freeze-dried formulation (cake or powder) of a protein/antibody, into a clear liquid.

Sugar/s herein include sugars and sugar alcohols such as polyols. Sugars can be referred to monosaccharides, disaccharides, and polysaccharides. Examples of sugars include, but are not limited to, sucrose, maltose, trehalose, glucose, dextrose, raffinose and others. Examples of polyols include, but are not limited to, mannitol, sorbitol, and others.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

Examples of salts include, but not limited to, sodium chloride, potassium chloride, magnesium chloride, sodium thiocyanate, ammonium thiocyanate, ammonium sulfate, ammonium chloride, calcium chloride, zinc chloride and/or sodium acetate.

Certain specific aspects and embodiments of the invention are more fully described by reference to the following examples. However, these examples should not be construed as limiting the scope of the invention in any manner.

### DETAILED DESCRIPTION OF EMBODIMENTS

The present invention discloses a method for obtaining a freeze-dried formulation of an anti-α4β7 formulation, which is stable for at least 3 months at room-temperature before reconstitution, and stable for at least 24 hours at room-temperature post reconstitution. Water for injection is used for reconstituting the freeze-dried antibody.

The freeze-dried anti-α4β7 antibody product obtained from the present invention exhibits stability under accelerated stability conditions such as at 40° C. for 4 weeks.

In an embodiment, the invention discloses a method of obtaining a room-temperature stable, reconstituted freeze-dried formulation of anti-α4β7 antibody, using a lyophilization method comprising steps of; preparation of a liquid anti-α4β7 antibody formulation, primary drying of the antibody formulation in the lyophilization method at the glass transition temperature of the formulation and holding it at the glass transition temperature, followed by increasing the temperature to a second and third drying temperatures and holding at the respective temperatures.

In the above mentioned embodiment, the disclosed freeze-dried anti-α4β7 antibody formulation remains stable for at least 24 hours at room-temperature, post reconstitution.

In an embodiment, the invention discloses a method of obtaining freeze-dried anti-α4β7 antibody composition using a lyophilization method, comprising preparation of a liquid anti-α4β7 antibody composition and subjecting the antibody composition to a lyophilization method comprising the steps of:
primary drying of the antibody composition at different temperatures which includes subjecting the antibody composition to a first temperature which is at the glass transition temperature of the antibody composition and holding it at the glass transition temperature, followed by increasing the temperature to a second and third drying temperatures and holding at the respective temperatures,
followed by secondary drying to obtain a freeze dried antibody composition and wherein, the freeze-dried antibody obtained using the said method remains stable for 24 hours at room temperature, post reconstitution.

In another embodiment, the invention discloses a method of obtaining a room-temperature stable, reconstituted freeze-dried formulation of anti-α4β7 antibody, using a lyophilization method comprising steps of:
preparation of liquid anti-α4β7 antibody in a composition comprising a sugar, amino acid and surfactant,
freezing the liquid antibody composition at a temperature, ranging from about −45° C. to about −50° C.,
annealing the frozen antibody composition at a temperature, ranging from about −22° C. to about −25° C.,
refreezing the antibody composition at a temperature, ranging from about −45° C. to about −50° C.,
primary drying of the antibody composition, in a step-wise approach by subjecting to different temperatures ranging from about −25° C. to about 0° C., wherein the first temperature in the step-wise approach is carried out at the glass transition (Tg) temperature of the composition which is −28° C. and holding it for a period of time, followed by increasing the temperature to −15° C. and holding it for another period of time, followed by further increasing the temperature to −10° C. and holding it for a period of time for followed by further increasing the temperature to 0° C.
secondary drying of the primary dried antibody composition, at a temperature ranging from about 10° C. to about 25° C. to obtain freeze-dried antibody composition, and wherein, the freeze-dried antibody composition obtained using the said steps remains stable for 24 hours at room temperature, post reconstitution.

In the above mentioned embodiment, the liquid anti-α4β7 antibody composition comprises at least 60 mg of anti-α4β7 antibody, 70 mg trehalose, at least 10 mg arginine, at least 2.5 mg NaCl and 0.6 mg polysorbate in 1 ml of 20 mM phosphate-histidine buffer composition having pH of about 6.0 to 6.5.

In an embodiment, the invention discloses a freeze-dried formulation of anti-α4β7 antibody comprising; 300 mg of anti-α4β7 antibody, 5 mg phosphate, 60 mg arginine, 375 mg trehalose, 15 mg sodium chloride, and 3 mg polysorbate 80 and wherein the freeze-dried formulation is stable at room-temperature for three months before reconstitution and, stable for at least 24 hours post reconstitution.

In yet another embodiment, the invention discloses a method for preparing a high-concentration freeze-dried formulation of anti-α4β7 antibody, comprising preparation of high-concentration anti-α4β7 antibody formulation comprising, at least 50 mg of anti-α4β7 antibody in 1 ml of phosphate-histidine buffer composition comprising trehalose, arginine, sodium chloride and polysorbate; freezing the said high-concentration anti-α4β7 antibody formulation at a temperature, ranging from about −45° C. to about −50° C., to transform the liquid formulation into a frozen state, annealing the frozen formulation obtained from the above freezing step at a temperatures ranging from about −22° C. to about −25° C., refreezing the formulation obtained from the above step at a temperature, ranging from about −45° C. to about −50° C., followed by primary drying of the formulation in a step-wise approach at different temperatures, ranging from about −25° C. to about 0° C. and wherein the first temperature in the step-wise approach is the glass transition (Tg) temperature of the formulation which is −28° C. and holding it for a period of time followed by increasing the temperature to −15° C. and holding it for another period of time followed by further increasing the temperature to −10° C. and holding it for followed by further increasing the temperature to 0° C.; secondary drying of the formulation obtained from the primary drying step, at a temperature ranging from about 10° C. to about 25° C., to produce a dry cake; and wherein the dry cake can be reconstituted with a diluent in about 15 minutes or less to produce a high-concentration reconstituted formulation and the reconstituted formulation exhibits stability for at least 24 hours at room temperature.

In another embodiment, the invention discloses a method of obtaining a room-temperature stable, freeze-dried anti-α4β7 antibody formulation comprising:
expressing and purifying anti-α4β7 antibody,
subjecting the purified antibody to one or more ultrafiltration and/or diafiltration steps with a buffer comprising addition of sugar and/or amino acid in the filtration steps,
formulating the antibody solution, obtained from the filtration step, with suitable excipients, and,
freeze-drying the antibody formulation by a lyophilization process,
   wherein the freeze-dried antibody formulation obtained by the method is stable at room temperature for at least 24 hours, after reconstitution and contains less than 1% of aggregate content (pre and post the lyophilization process), and wherein, the freeze-dried powder/cake is stable and free from collapse and contains less than 1% moisture content.

In the above mentioned embodiment, the lyophilization process comprises steps of: freezing the antibody formulation comprising a sugar, amino acid and surfactant at a temperature, ranging from about −45° C. to about −50° C., to transform the liquid formulation into a frozen state, annealing the frozen formulation at a temperature, ranging from about −22° C. to about −25° C., refreezing the formulation obtained from the above step at a temperature, ranging from about −45° C. to about −50° C., primary drying of the re-freezed formulation, at temperatures, ranging from about −25° C. to about 0° C. and wherein the first temperature of primary drying is the glass transition (Tg) temperature of the formulation which is −28° C. and holding it for a period of time followed by increasing the temperature to −15° C. and holding it for another period of time followed by further increasing the temperature to −10° C. and holding it for followed by further increasing the temperature to 0° C., secondary drying of the primary dried formulation, at a temperature ranging from about 10° C. to about 25° C.

In any of the above mentioned embodiments, freezing rate maintained during the lyophilization process except secondary drying is about 0.2-0.4° C./minute, preferably 0.3° C./minute.

In any of the above mentioned embodiments, the primary drying step is performed at a pressure range from 100±20 μBar.

In any of the above mentioned embodiments, the freeze-dried anti-α4β7 antibody formulation is stable and contains less than 1% of the aggregate content when stored at 25° C. for three months and less than 0.5% when stored at 2-8° C. The aggregate content of less than 0.5% is maintained at least for 6 months when stored at 2-8° C.

In any of the above mentioned embodiments, the freeze-dried, anti-α4β7 antibody is reconstituted in less than 10 minutes, preferably in less than 5 minutes, more preferably in less than 3 minutes.

In any of the above mentioned embodiments, the freeze-dried, anti-α4β7 antibody present in the formulation is biologically active.

In any of the above mentioned embodiments, the pH of the anti-α4β7 antibody formulation is 6.0.

In yet another embodiment, the invention discloses a method for obtaining freeze-dried formulation of vedolizumab, comprising preparation of vedolizumab liquid formulation comprising at least 60 mg vedolizumab, 75 mg trehalose, 12 mg arginine and 0.6 mg polysorbate-80 in 20 mM histidine-phosphate buffer, subjecting the liquid formulation to lyophilization process, wherein the lyophilization process comprises steps of, primary drying of antibody formulation in a step-wise approach, at different temperatures ranging from −25° C. to 0° C., the first temperature in this step-wise approach is subjecting the antibody formulation to glass transition temperature (Tg) of the formulation and holding at the glass transition temperature followed by increasing temperatures to second and third temperatures and holding at the respective temperatures followed by secondary drying to obtain a freeze-dried antibody formulation and wherein, the freeze-dried formulation is stable at room temperature for at least 24 hours, after reconstitution and contains less than 1% of aggregate content (pre and post the lyophilization process), and wherein, the freeze-dried powder/cake is stable and free from collapse and contains less than 1% moisture content.

In any of the above mentioned embodiments, the formulation exhibits stability at 40° C. for at least four weeks, wherein the percentage of aggregate content is less than 1.5% after storage at 40° C. for four weeks.

In any of the above mentioned embodiments, the reconstituted formulation is stable without any visible particles.

In an embodiment, the invention discloses a method of lyophilization to obtain a freeze-dried anti-α4β7 antibody composition that maintains the quality attributes of the anti-α4β7 antibody composition as same, before and after the lyophilization method, the method comprising steps of, subjecting a liquid anti-α4β7 antibody composition to primary drying in a step-wise approach at different temperatures, wherein the antibody solution is subjected to a/first temperature which is glass transition temperature (Tg) of the antibody composition and holding at the glass transition temperature, followed by increasing temperatures to second and third temperatures and holding at the respective temperatures, followed by secondary drying of the antibody to obtain a freeze-dried antibody composition.

In the embodiment, the quality attributes of the antibody include, aggregate content, fragment content, charge variants content and main peak content. The present invention is specifically advantageous in terms of storage of the therapeutic antibody, in particular, storage of the freeze-dried anti-α4β7 antibody, post reconstitution with water. The antibody can be stored at room temperature which is essential when 2-8° C. conditions are not available to patients or healthcare professionals. Further, the disclosed lyophilisation cycle is accomplished in less than 72 and preferably in less than 69 hours.

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

An anti-α4β7 antibody molecule, vedolizumab, suitable for storage in the present pharmaceutical composition is produced by standard methods known in the art. For example, vedolizumab is prepared by recombinant expression of vedolizumab in a mammalian host cell such as Chinese Hamster Ovary cells. Further, the expressed vedolizumab is harvested and the crude harvest is subjected to standard downstream process steps that include purification, filtration and optionally dilution or concentration steps. For example, the crude harvest of vedolizumab may be purified using standard chromatography techniques such as affinity chromatography, ion-exchange chromatography and combinations thereof. The purified vedolizumab solution can additionally be subjected to one or more filtration steps, and the solution obtained is subjected to further formulation studies.

### Example-1: Freeze-Dried Vedolizumab Formulations

8-15 mg/ml concentration of vedolizumab in acetate buffer obtained from chromatographic step was subjected for ultrafiltration to concentrate up to 60 mg/ml. Post which, the samples were subjected for diafiltration wherein the diafiltration medium contained 20 mM histidine-phosphate buffer (formulation buffer) with excipients such as at least 60 mg/ml trehalose, amino acid and/or salt. Post diafiltration, the samples were subjected for second ultrafiltration to concentrate up to 60 mg/ml to 80 mg/ml. Post which, polysorbate −80 was added. Approved vedolizumab lyophilization formulation contains 100 mg/ml sucrose, 26.2 mg/ml arginine and 0.6 mg/ml polysorbate 80. To maintain positive control, 100 mg/ml sucrose and arginine were added during ultrafiltration. Details of the final formulations are given Table 1. Post which, glass transition temperature of various formulations were measured using modulated differential scanning calorimetry (DSC) and the same Tg values had been selected as the first temperature of primary drying in step wise approach during lyophilization. All samples were filed in vials and subjected for lyophilisation process. Details of the lyophilisation process is given in Table 2. Post lyophilisation, all the samples were kept at 2-8° C. for 3 months, 40° C. for 2 months or 25° C. for 3 months and checked for various quality attributes such as pH, appearance of cake, high molecular weight content (HMW) and moisture content. The quality attributes were checked at different time points when stored at different temperatures. HMW content was measured using size exclusion chromatography and moisture content was measured using Fourier Transform Near infrared spectroscopy. Result of the quality attributes of freeze dried formulations are given in Table 3.

TABLE 1
Details of various vedolizumab formulations prior
to lyophilization prepared as per Example 1.

| Sample | Formulated Drug substance composition (prior to |
|---|---|
| ID | lyophilization) |
| Vmab- | Vedolizumab 60 mg/ml, 50 mM histidine, 26.3 mg/ml arginine, |
| Control | sucrose 100 mg/ml, 0.6 mg/ml polysorbate 80 |
| Vmab-1 | Vedolizumab 60 mg/ml, 20 mM histidine-phosphate, 12 mg/ml |
| | arginine, 2.92 mg/ml sodium chloride, 0.6 mg/ml polysorbate 80 |
| Vmab-2 | Vedolizumab 60 mg/ml, 20 mM histidine-phosphate, 9 mg/ml |
| | arginine, 1 mg/ml glycine, 0.6 mg/ml polysorbate 80 |
| Vmab-3 | Vedolizumab 60 mg/ml, 20 mM histidine-phosphate, 8 mg/ml |
| | lysine, 1 mg/ml glycine, 0.6 mg/ml polysorbate 80 |

TABLE 2
Lyophilization cycle for vedolizumab lyophilized product

| Process parameter | In-Process control |
|---|---|
| Loading temperature | 5 ± 3° C. |
| Freezing (ramp) | 5° C. to −45° C. for NLT 2.5 hours |
| Freezing (hold) | −45° C. for 4 to 8 hours |
| Annealing | −22° C. for 3 to 6 hours |
| Refreezing (ramp) | −45° C. for NLT 1 hour |
| Refreezing (hold) | −45° C. for NLT 4 hours |
| Primary drying (Ramp) | −28° C. for NLT 0.5 hours at 100 ± 20 μbar |
| | pressure |
| Primary drying (hold) | −28° C. for NLT 3 hours at 100 ± 20 μbar |
| | pressure |
| Primary drying (Ramp) | −15° C. for NLT 0.5 hour at 100 ± 20 μbar |
| | pressure |
| Primary drying (hold) | −15° C. for NLT 3 hours at 100 ± 20 μbar |
| | pressure |
| Primary drying (Ramp) | −10° C. for NLT 0.15 hour at 100 ± 20 μbar |
| | pressure |
| Primary drying (hold) | −10° C. for NLT 28 hours at 100 ± 20 μbar |
| | pressure |
| Primary drying (Ramp) | 0° C. for NLT 0.5 hour at 100 ± 20 μbar |
| | pressure |
| Primary drying (hold) | 0° C. for NLT 8 hours at 100 ± 20 μbar |
| | pressure |
| Secondary drying (Ramp) | 25° C. for NLT 2.5 hours at 750 ± 20 μbar |
| | pressure |
| Secondary drying (hold) | 25° C. for NLT 8 hours at 750 ± 20 μbar |
| | pressure |
| Stoppering | 25° C. at 750 ± 20 μbar pressure |

NLT—not less than

TABLE 3
Quality attributes of various freeze dried formulations
prepared as per example 1 at 40° C. for two months

| | | | Reconstitution | | |
|---|---|---|---|---|---|
| | pH | | time (in minutes) | | Moisture content |
| Sample ID | T0 | T2M | T0 | T2M | (%) |
| Vmab- | 6.3 | 6.4 | 7.35 | 6.37 | 0.8 |
| control | | | | | |
| Vmab-1 | 6.2 | 6.2 | 8.38 | 5.43 | 1.0 |
| Vmab-2 | 6.2 | 6.2 | 8.25 | 6.38 | N/A |
| Vmab-3 | 6.2 | 6.2 | 8.11 | 6.22 | N/A |

M—represents months; N/A—not applicable and not measured due to sample constrain.

TABLE 4
High molecular weight species and main peak content of vedolizumab freeze dried
CTLA4-Ig fusion protein formulation when stored at 40° C. for one/two month

| | HMW | | Main peak content | | Acidic variants | | Basic variants | |
|---|---|---|---|---|---|---|---|---|
| | | 40° C. | | 40° C. | | 40° C. | | 40° C. |
| Sample ID | T0 | T2M | T0 | T1M | T0 | T1M | T0 | T1M |
| Vmab-control | 0.3 | 0.4 | 71.4 | 71.4 | 20.1 | 19.8 | 8.5 | 10.3 |
| Vmab-1 | 0.4 | 1.1 | 71.2 | 71.2 | 19.3 | 20.1 | 9.6 | 11.6 |
| Vmab-2 | 0.5 | 1.6 | 70.6 | 70.6 | 19.3 | 20.2 | 10.1 | 12.1 |
| Vmab-3 | 0.5 | 2.0 | 70.7 | 70.7 | 20.5 | 20.3 | 8.8 | 12.6 |

M—represents months
‘No major changes were observed in monomer content and acidic/basic variants of the samples when stored at 25° C. for one month and 40° C. for one month.
Biological activity of Vmab control and Vmab-1 sample was measured after storing the sample at 40° C. for one month.

TABLE 5
Biological activity of vedolizumab
samples prepared as per Example 1

Biological activity at 40° C.

| Sample ID | T0 | T1M |
|---|---|---|
| Vmab control | 98 | 98 |
| Vmab-1 | 94 | 104 |

TABLE 6
Quality attributes of vedolizumab formulations prepared
as per example 1 when stored at 25° C. for three months

| | | | | | | | % main |
|---|---|---|---|---|---|---|---|
| Sample | | Visual | Cake | Reconstitution | | Monomer | peak |
| ID | pH | appearance | appearance | time | HMW | Content | content |
| Vmab-C | 6.3 | Opalescent, | White, intact | 2.30 | 0.28 | 99.6 | 70.57 |
| Vmab-1 | 6.2 | colour less | cake | 3.20 | 0.58 | 99.3 | 69.91 |
| Vmab-2 | 6.1 | and no | Fragmented | 3.18 | 0.80 | 99.1 | 69.96 |
| | | visible | cake | | | | |
| Vmab-3 | 6.2 | particle | White intact | 3.26 | 0.87 | 99.03 | 69.70 |
| | | | cake | | | | |

TABLE 7
Quality attributes of vedolizumab formulations prepared
as per example 1 when stored at 2-8° C. for three months

| | | | | | | | % main |
|---|---|---|---|---|---|---|---|
| Sample | | Visual | Cake | Reconstitution | | Monomer | peak |
| ID | pH | appearance | appearance | time | HMW | Content | content |
| Vmab-C | 6.3 | Opalescent, | White, intact | 6.08 | 0.28 | 99.64 | 70.75 |
| | | colour | cake | | | | |
| Vmab-1 | 6.2 | less and | | 3.15 | 0.38 | 99.56 | 70.35 |
| Vmab-2 | 6.1 | no visible | White, intact | 3.13 | 0.48 | 99.31 | 70.18 |
| | | particles | cake | | | | |
| Vmab-3 | 6.2 | | White, intact | 3.25 | 0.61 | 99.19 | 69.90 |
| | | | cake | | | | |

### Example 2: Room Temperature Stability of Reconstituted Vedolzumab Formulations

All vedolizumab samples prepared in Example 1 were kept at 2-8° C. for six months. Post which, the samples were reconstituted with sterile water for injection and kept at room temperature for 24 hours. Post which, the samples were checked for various quality attributes such as change in pH, monomer content, aggregate content/high molecular weight species using size exclusion chromatography and also main peak content, acidic variants/basic variants using ion exchange chromatography. Further, the data was compared with initial data points as well as samples which were reconstituted immediately after storage at 2-8° C. for six months. The results are given in below Table 8 and 9.

TABLE 8
SEC data of vedolizumab samplers prepared as example 2

| | % HMW | | | % monomer | | |
|---|---|---|---|---|---|---|
| | | | 2-8 6 M | | | 2-8 6 M |
| Sample ID | T0 | 2-8 6 M | (RT 24 H) | T0 | 2-8 6 M | (RT 24 H) |
| Vmab-Control | 0.3 | 0.2 | 0.3 | 99.7 | 99.8 | 99.7 |
| Vmab-1 | 0.4 | 0.4 | 0.4 | 99.6 | 99.6 | 99.6 |
| Vmab-2 | 0.5 | 0.6 | 0.5 | 99.5 | 99.4 | 99.5 |
| Vmab-3 | 0.5 | 0.5 | 0.6 | 99.5 | 99.5 | 99.4 |

TABLE 9
IEX data of vedolizumab samplers prepared as example 2

| | % Acidic variants | | | % Main peak content | | |
|---|---|---|---|---|---|---|
| | | | 2-8 6 M | | | 2-8 6 M |
| Sample ID | T0 | 2-8 6 M | (RT 24 H) | T0 | 2-8 6 M | (RT 24 H) |
| Vmab-C | 20.1 | 22.1 | 22.2 | 71.4 | 69.5 | 69.1 |
| Vmab-1 | 19.3 | 23.4 | 22.6 | 71.2 | 67.5 | 68.7 |
| Vmab-2 | 19.3 | 23.8 | 23.1 | 70.6 | 67.3 | 68.2 |
| Vmab-3 | 20.5 | 24.2 | 22.5 | 70.7 | 66.8 | 68.6 |

Further, these samples were checked for change in pH and osmolality. It has been observed that, there was no major change in pH and osmolality.

1. A method of obtaining a freeze-dried anti-α487 antibody composition using a lyophilization method, comprising preparation of a liquid anti-α487 antibody composition and subjecting the antibody composition to a lyophilization method comprising the steps of:
primary drying of the antibody composition at different temperatures which includes subjecting the antibody composition to a first temperature which is at the glass transition temperature of the antibody composition and holding it at the glass transition temperature, followed by increasing the temperature to a second and third drying temperatures, and holding it at the respective temperatures,
followed by secondary drying to obtain a freeze dried antibody composition and wherein, the freeze-dried antibody obtained using the said method remains stable for 24 hours at room temperature, post reconstitution.

2. A method of obtaining a room-temperature stable, reconstituted freeze-dried formulation of anti-α487 antibody, using a lyophilization method comprising steps of:
preparation of liquid anti-α487 antibody in a composition comprising a sugar, amino acid and surfactant,
freezing the liquid antibody composition at a temperature, ranging from about −45° C. to about −50° C.,
annealing the frozen antibody composition at a temperature, ranging from about −22° C. to about −25° C.,
refreezing the antibody composition at a temperature, ranging from about −45° C. to about −50° C.,
primary drying of the antibody composition, in a step-wise approach by subjecting to different temperatures ranging from about −25° C. to about 0° C., wherein the first temperature in the step-wise approach is carried out at the glass transition (Tg) temperature of the composition which is −28° C. and holding it for a period of time, followed by increasing the temperature to −15° C. and holding it for another period of time, followed by further increase of temperature to −10° C. and holding it for a period of time, followed by a further increase of temperature to 0° C., and,
secondary drying of the primary dried antibody composition, at a temperature ranging from about 10° C. to about 25° C. to obtain freeze-dried antibody composition, and wherein, the freeze-dried antibody composition obtained using the said steps remains stable for 24 hours at room temperature, post reconstitution.

3. The method according to claim 1, wherein the liquid anti-α4β7 antibody composition comprises at least 60 mg of anti-α4β7 antibody, at least 70 mg trehalose, at least 10 mg arginine, at least 2.5 mg NaCl and 0.6 mg polysorbate in 1 ml of 20 mM phosphate-histidine buffer composition having pH of about 6.0 to 6.5.

4. The method according to claim 1, wherein the primary drying step is performed at a pressure range from 100±20 μBar.

5. The method according to claim 1, wherein the freeze-dried anti-α4β7 antibody composition remains stable and contains less than 1% of the aggregate content when stored at 25° C. for three months and less than 0.5% of the aggregate content when stored at 2-8° C. for six months.

6. The method according to claim 1, wherein the freeze-dried anti-α4β7 antibody formulation is reconstituted in less than 10 minutes.

7. The method according to claim 1 wherein the freeze-dried anti-α4β7 antibody formulation is stable and free from collapse and contains less than 1% moisture content.

8. A stable formulation of anti-α4β7 antibody comprising, 60 mg/ml vedolizumab antibody, histidine containing buffer, 60 mg/ml to 75 mg/ml trehalose, one or more amino acids and surfactant, wherein the formulation is a liquid or a reconstituted freeze-dried formulation.

9. The composition according to claim 2, wherein the amino acid is arginine, glycine, or lysine, or combinations thereof.

10. The formulation according to claim 8, which further comprises a salt.

11. A method of obtaining a room-temperature stable, freeze-dried anti-α4β7 antibody formulation comprising:
expressing and purifying anti-α4β7 antibody,
subjecting the purified antibody to one or more ultrafiltration and/or diafiltration steps with a buffer comprising addition of sugar and/or amino acid in the filtration steps, formulating the antibody solution, obtained from the filtration step, with suitable excipients, and,
freeze-drying the antibody formulation by a lyophilization process,
wherein the freeze-dried antibody formulation obtained by the method is stable at room temperature for at least 24 hours, after reconstitution and contains less than 1% of aggregate content (pre and post the lyophilization process), and wherein the freeze-dried powder/cake is stable and free from collapse and contains less than 1% moisture content.

12. The method according to claim 2, wherein the liquid anti-α4β7 antibody composition comprises at least 60 mg of anti-α4β7 antibody, at least 70 mg trehalose, at least 10 mg arginine, at least 2.5 mg NaCl and 0.6 mg polysorbate in 1 ml of 20 mM phosphate-histidine buffer composition having pH of about 6.0 to 6.5.

13. The method according to claim 2, wherein the primary drying step is performed at a pressure range from 100±20 μBar.

14. The method according to claim 2, wherein the freeze-dried anti-α4β7 antibody composition remains stable and contains less than 1% of the aggregate content when stored at 25° C. for three months and less than 0.5% of the aggregate content when stored at 2-8° C. for six months.

15. The method according to claim 2, wherein the freeze-dried anti-α4β7 antibody formulation is reconstituted in less than 10 minutes.

16. The method according to claim 2 wherein the freeze-dried anti-α4β7 antibody formulation is stable and free from collapse and contains less than 1% moisture content.
